(19) United States
(12) Patent Application Publication
(10) Pub. No.: US 2025/0001121 A1
(43) Pub. Date: Jan. 2, 2025

(54) LIQUID RESERVOIR

(71) Applicant: HCMed Innovations Co., LTD., Taipei City (TW)

(72) Inventors: Chieh-Sheng Cheng, Taipei City (TW); CHUN-CHIA JUAN, New Taipei City (TW); CHIA-CHIEN CHANG, New Taipei City (TW); YEN-TING CHEN, Taipei City (TW)

(21) Appl. No.: 18/757,698

(22) Filed: Jun. 28, 2024

Related U.S. Application Data
(60) Provisional application No. 63/524,193, filed on Jun. 29, 2023.

(30) Foreign Application Priority Data
Jun. 21, 2024 (CN) 202410805420.0

(51) Int. Cl. A61M 16/14; A61M 11/00; A61M 16/00

(52) U.S. Cl. CPC A61M 16/14; A61M 11/005; A61M 16/0003; A61M 2209/06

(57) ABSTRACT

A liquid reservoir includes a liquid storage chamber, a nebulizing module, a detection module, a lid, and a bubble blocking structure. The liquid storage chamber has an opening, and a bottom of the liquid storage chamber has a through hole. The nebulizing module is disposed in the through hole. The detection module is disposed within the liquid storage chamber and adjacent to the through hole. The lid is disposed on the liquid storage chamber and covers the opening. The bubble blocking structure is disposed in the liquid storage chamber, and an orthogonal projection of the bubble blocking structure that is projected to the bottom of the liquid storage chamber at least partially overlaps with the through hole.

### CROSS-REFERENCE TO RELATED PATENT APPLICATION

This application claims the benefit of priority to the U.S. Provisional Patent Application Ser. No. 63/524,193 filed on Jun. 29, 2023, which application is incorporated herein by reference in its entirety.

This application claims the benefit of priority to China Patent Application No. 202410805420.0, filed on Jun. 21, 2024 in People's Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a liquid reservoir, and more particularly to a liquid reservoir with a bubble blocking structure.

### BACKGROUND OF THE DISCLOSURE

Nebulizers for medical use mainly deliver drugs through the respiratory system of a user's body. The user pours an amount of medicinal liquid into a liquid reservoir inside the nebulizer. A nebulizing unit of the nebulizer can nebulize the medicinal liquid into tiny aerosol particles with a specific particle size and deliver them from the user's mouth and nose into the respiratory system of the user's body through breathing for treatment purposes.

An existing nebulizer can utilize a detection unit disposed inside the liquid reservoir to detect the amount of medicinal liquid in the liquid reservoir. The detection unit can detect and determine whether or not the medicinal liquid in the liquid reservoir is about to be used up, and output a corresponding detection signal to a control unit in the nebulizer to stop the operation of the nebulizing unit in advance, thereby preventing the nebulizing unit from continuing to vibrate and causing damage when the medicinal liquid is used up.

However, some medicinal liquid generate bubbles during the vibration process by the nebulizing unit, and the bubbles cover or remain on the detection unit, so as to affect the sensing result of the detection unit. Therefore, the detection unit cannot accurately detect the amount of the medicinal liquid and control the operation of the nebulizing unit accordingly.

### SUMMARY OF THE DISCLOSURE

In response to the above-referenced technical inadequacy, the present disclosure provides a liquid reservoir with a bubble blocking structure.

In order to solve the above-mentioned problems, one of the technical aspects adopted by the present disclosure is to provide a liquid reservoir, which includes a liquid storage chamber, a nebulizing module, a detection module, a lid, and a bubble blocking structure. The liquid storage chamber has an opening, and a bottom of the liquid storage chamber has a through hole. The nebulizing module is disposed in the through hole. The detection module is disposed within the liquid storage chamber and adjacent to the through hole. The lid is disposed on the liquid storage chamber and covers the opening. The bubble blocking structure is disposed in the liquid storage chamber, and an orthogonal projection of the bubble blocking structure that is projected to the bottom of the liquid storage chamber at least partially overlaps with the through hole.

In order to solve the above-mentioned problems, another one of the technical aspects adopted by the present disclosure is to provide a liquid reservoir, which includes a liquid storage chamber, a nebulizing module, a detection module, a lid, and a bubble blocking structure. The liquid storage chamber has an opening, and a bottom of the liquid storage chamber has a through hole. The nebulizing module is disposed in the through hole. The detection module is disposed within the liquid storage chamber and adjacent to the through hole. The lid is disposed on the liquid storage chamber and covers the opening. The bubble blocking structure is disposed in the liquid storage chamber. The bubble blocking structure includes a plurality of annular members surrounding the through hole and jointly forming a structure of concentric circles.

In order to solve the above-mentioned problems, yet another one of the technical aspects adopted by the present disclosure is to provide a liquid reservoir, which includes a liquid storage chamber, a nebulizing module, a detection module, a lid, and a bubble blocking structure. The liquid storage chamber has an opening, and a bottom of the liquid storage chamber has a through hole. The nebulizing module is disposed in the through hole. The detection module is disposed within the liquid storage chamber and adjacent to the through hole. The lid is disposed on the liquid storage chamber and covers the opening. The bubble blocking structure is disposed in the liquid storage chamber. The bubble blocking structure includes a straight pipe, and the straight pipe is located directly above the through hole. The straight pipe does not contact the nebulizing module or the through hole, but collects bubbles generated during the nebulizing process of the medicinal liquid above the through hole.

Therefore, in the liquid reservoir provided by the present disclosure, by the bubble blocking structure being disposed within the liquid storage chamber, the bubble blocking structure can be used to either adhere to or attract bubbles through surface properties, or to block the bubbles through structural design. When the bubbles are generated during the nebulizing process of the medicinal liquid, the bubble blocking structure collects and isolates the bubbles to prevent them from covering or remaining in a detection area of the detection module, thereby avoiding any impact on the detection results caused by the bubbles covering or remaining in the detection area.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a liquid reservoir with a lid thereof being covered according to a first embodiment of the present disclosure;

FIG. 2 is a schematic view of the liquid reservoir with the lid thereof being open according to the first embodiment of the present disclosure;

FIG. 3 is a schematic cross-sectional view of the liquid reservoir according to the first embodiment of the present disclosure;

FIG. 4 is a schematic view of a liquid reservoir opening a lid thereof according to a second embodiment of the present disclosure;

FIG. 5 is a schematic cross-sectional view of the liquid reservoir according to the second embodiment of the present disclosure;

FIG. 6 is a schematic view of a liquid reservoir opening a lid thereof according to a third embodiment of the present disclosure;

FIG. 7 is a schematic cross-sectional view of the liquid reservoir according to the third embodiment of the present disclosure;

FIG. 8 is a schematic view of a liquid reservoir with a lid thereof being covered according to a fourth embodiment of the present disclosure;

FIG. 9 is a schematic view of the liquid reservoir opening the lid thereof according to the fourth embodiment of the present disclosure;

FIG. 10 is a schematic cross-sectional view of the liquid reservoir according to the fourth embodiment of the present disclosure;

FIG. 11 is a schematic view of a liquid reservoir covering a lid thereof according to a fifth embodiment of the present disclosure;

FIG. 12 is a schematic view of the liquid reservoir opening the lid thereof according to the fifth embodiment of the present disclosure;

FIG. 13 is a schematic cross-sectional view of the liquid reservoir according to the fifth embodiment of the present disclosure;

FIG. 14 is a schematic view of a liquid reservoir covering a lid thereof according to a sixth embodiment of the present disclosure;

FIG. 15 is a schematic view of the liquid reservoir opening the lid thereof according to the sixth embodiment of the present disclosure;

FIG. 16 is a schematic cross-sectional view of the liquid reservoir according to the sixth embodiment of the present disclosure;

FIG. 17 is a schematic view of a liquid reservoir opening a lid thereof according to a seventh embodiment of the present disclosure;

FIG. 18 is a partial schematic exploded view of the liquid reservoir according to the seventh embodiment of the present disclosure;

FIG. 19 is a schematic view of a liquid reservoir opening a lid thereof according to an eighth embodiment of the present disclosure;

FIG. 20 is a schematic view of a liquid storage chamber according to the eighth embodiment of the present disclosure;

FIG. 21 is a schematic view of a bubble blocking structure according to the eighth embodiment of the present disclosure;

FIG. 22 is a schematic view of a liquid reservoir covering a lid thereof according to a ninth embodiment of the present disclosure;

FIG. 23 is a schematic view of the liquid reservoir opening the lid thereof according to the ninth embodiment of the present disclosure;

FIG. 24 is a schematic cross-sectional view of the liquid reservoir according to the ninth embodiment of the present disclosure;

FIG. 25 is a schematic view of a liquid reservoir opening a lid thereof according to a tenth embodiment of the present disclosure;

FIG. 26 is a schematic view of a bubble blocking structure according to the tenth embodiment of the present disclosure;

FIG. 27 is a schematic view of a liquid reservoir opening a lid thereof according to an eleventh embodiment of the present disclosure;

FIG. 28 is a schematic view of a liquid reservoir opening a lid thereof according to a twelfth embodiment of the present disclosure; and

FIG. 29 is a schematic view of a liquid reservoir opening a lid thereof according to a thirteenth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

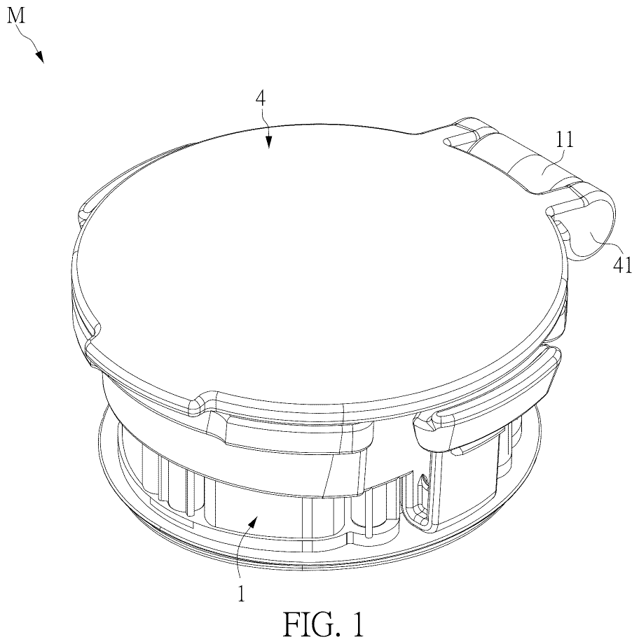

Reference is made to FIG. 1 to FIG. 3. A first embodiment of the present disclosure provides a liquid reservoir M suitable for a nebulizer (not shown in the figures), which includes a liquid storage chamber 1, a nebulizing module 2, a detection module 3, a lid 4, and a bubble blocking structure 5. The liquid storage chamber 1 is an accommodating cavity therein for storing medicinal liquid. A top of the liquid storage chamber 1 has an opening 101, and a bottom of the liquid storage chamber 1 has a through hole 102. The opening 101 and the through hole 102 are in spatial communication with each other through the accommodating cavity. The nebulizing module 2 is disposed in the through hole 102. The detection module 3 is disposed within the liquid storage chamber 1, and the detection module 3 is exposed from the accommodating cavity and adjacent to the through hole 102.

When the medicinal liquid is stored in the liquid storage chamber 1, the detection module 3 is immersed in the medicinal liquid, such that the detection module 3 can detect an amount of the medicinal liquid to determine whether or not the medicinal liquid in the liquid storage chamber 1 is about to be used up. The medicinal liquid passes through the through hole 102 and is nebulized by the nebulizing module 2 to form an aerosol, and the aerosol is sprayed into the user's mouth and nose through the nozzle (not shown in the figures) of the nebulizer. The lid 4 covers the opening 101. The lid 4 includes a pivot member 41, and the lid 4 is pivotally connected to a pivot shaft 11 of the liquid storage chamber 1 through the pivot member 41 for opening or closing.

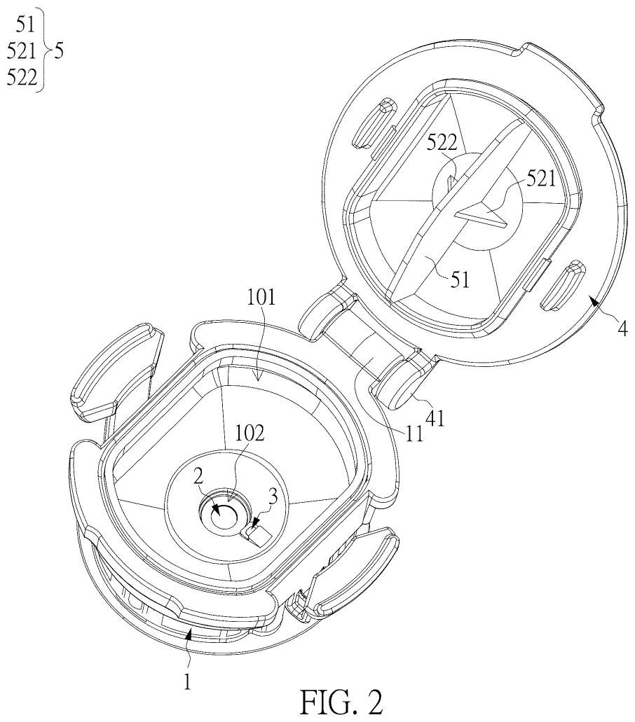

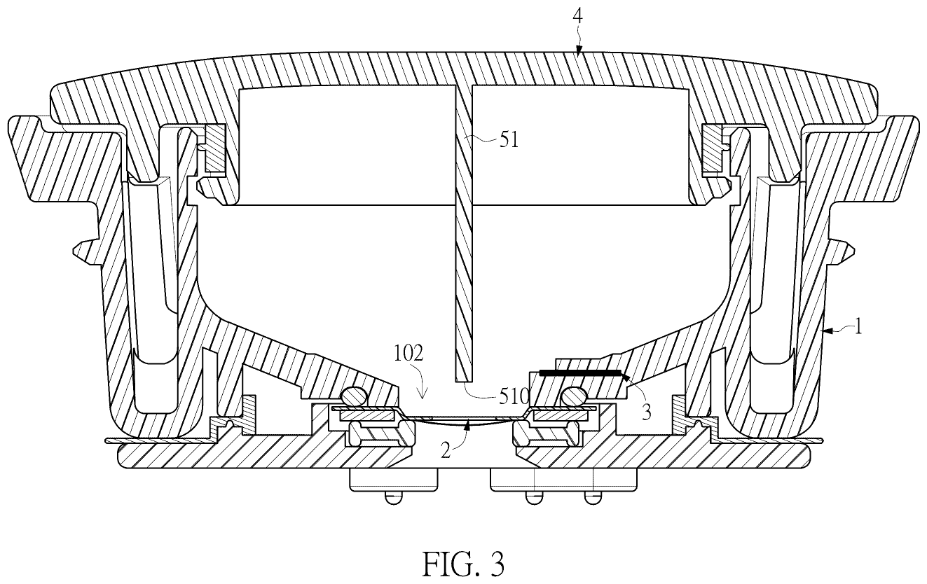

Reference is made to FIG. 2 and FIG. 3. The bubble blocking structure 5 includes a partition wall 51, and the partition wall 51 is connected to a lower surface of the bubble blocking structure 5 and extends toward the through hole 102. Two sides of the bubble blocking structure 51 are provided with a first fixing rib 521 and a second fixing rib 522, respectively. The first fixing rib 521 and the second fixing rib 522 are used to strengthen the structural strength of the partition wall 51.

When the lid 4 is covered, the bubble blocking structure 5 is disposed within the liquid storage chamber 1. Reference is made to FIG. 3. A lower edge 510 of the bubble blocking structure 5 is lower than the detection module 3 and higher than the through hole 102. The lower edge 510 and the through hole 102 have a gap therebetween. Preferably, the gap ranges between 0.2 mm and 10 mm. An orthogonal projection of the partition wall 51 of the bubble blocking structure 5 that is projected onto the bottom of the liquid storage chamber 1 at least partially overlaps the through hole 102. Specifically, the orthogonal projection of the partition wall 51 that is projected onto the bottom of the liquid storage chamber 1 is located at a central position of the through hole 102.

Therefore, the partition wall 51 can be used to either adhere to or attract bubbles through surface properties, or to block the bubbles through structural design. When the bubbles are generated during the nebulizing process of the medicinal liquid, the partition wall 51 block the bubbles to prevent them from covering or remaining in a detection area of the detection module 3, thereby avoiding any impact on the detection results caused by the bubbles covering or remaining in the detection area.

However, the aforementioned description for the bubble blocking structure 5 of the first embodiment is merely an example, and is not meant to limit the scope of the present disclosure. The subsequent embodiments are used to illustrate variations of the bubble blocking structure 5. The structures of the liquid storage chamber 1, the nebulizing module 2, the detection module 3, and the lid 4 in the liquid reservoir M of the subsequent embodiments are similar to those of the first embodiment.

### Second Embodiment

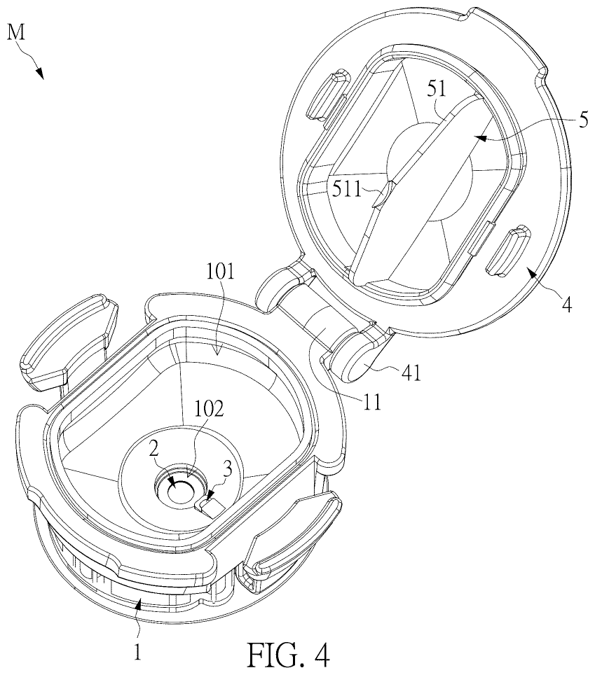

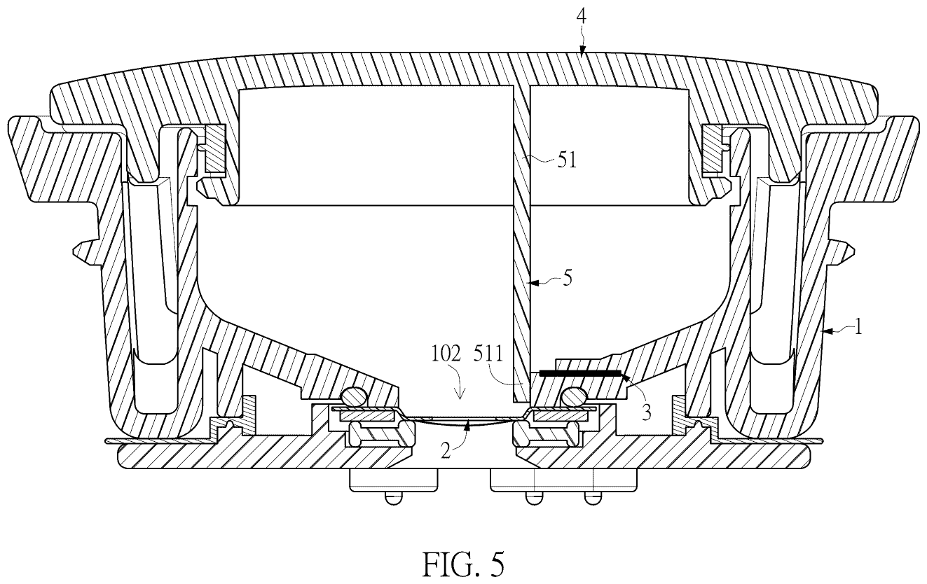

Reference is made to FIG. 4 and FIG. 5. A second embodiment of the present disclosure provides a liquid reservoir M, which includes a liquid storage chamber 1, a nebulizing module 2, a detection module 3, a lid 4, and a bubble blocking structure 5. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 is pivotally connected to the liquid storage chamber 1 and covers the opening 101.

The bubble blocking structure 5 includes a partition wall 51, and the partition wall 51 is connected to a lower surface of the bubble blocking structure 5. An orthogonal projection of the partition wall 51 that is projected onto the bottom of the liquid storage chamber 1 is located between the detection module 3 and a central position of the through hole 102. Comparing FIG. 5 with FIG. 3, the partition wall 51 of the second embodiment is closer to the detection module 3 than that of the first embodiment. Preferably, a lower edge of the partition wall 51 includes a convex portion 511, and the convex portion 511 extends into the through hole 102 and abuts against an inner edge of the through hole 102.

### Third Embodiment

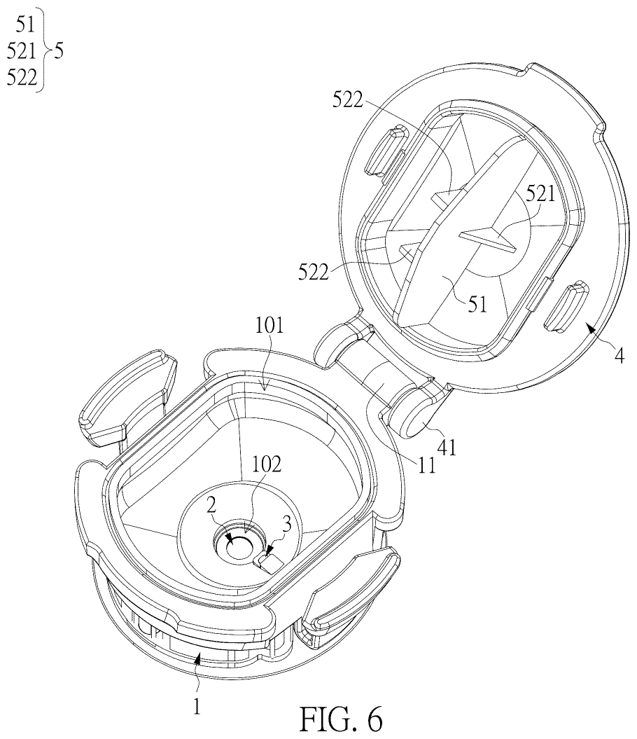

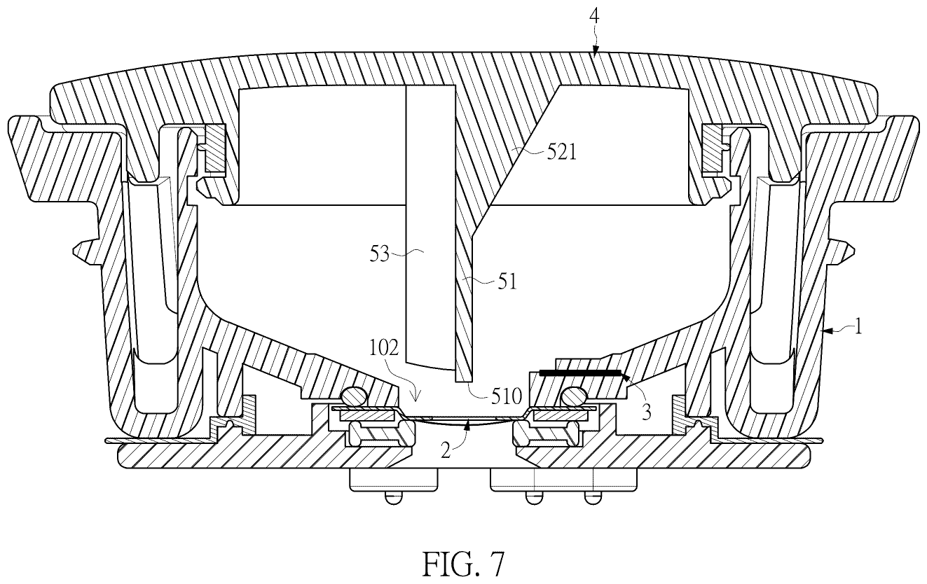

Reference is made to FIG. 6 and FIG. 7. A third embodiment of the present disclosure provides a liquid reservoir M, which includes a liquid storage chamber 1, a nebulizing module 2, a detection module 3, a lid 4, and a bubble blocking structure 5. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 is pivotally connected to the liquid storage chamber 1 and covers the opening 101.

The bubble blocking structure 5 includes a partition wall 51, and the partition wall 51 is connected to a lower surface of the bubble blocking structure 5. One side of the partition wall 51 is provided with a first fixing rib 521, and another side of the partition wall 51 is provided with two second fixing ribs 522. The two second fixing ribs 522 are orthogonally projected onto the bottom of the liquid storage chamber 1 to form two second orthogonal projections, and the through hole 102 is located between the two second orthogonal projections. The first fixing rib 521 and the two second fixing ribs 522 are used to strengthen the structural strength of the partition wall 51. A lower edge 510 of the partition wall 51 is lower than the detection module 3 and higher than the through hole 102. The lower edge 510 and the through hole 102 have a gap therebetween. Preferably, the gap ranges between 0.2 mm and 10 mm. An orthogonal projection of the partition wall 51 that is projected onto the bottom of the liquid storage chamber 1 is located at a central position of the through hole 102.

### Fourth Embodiment

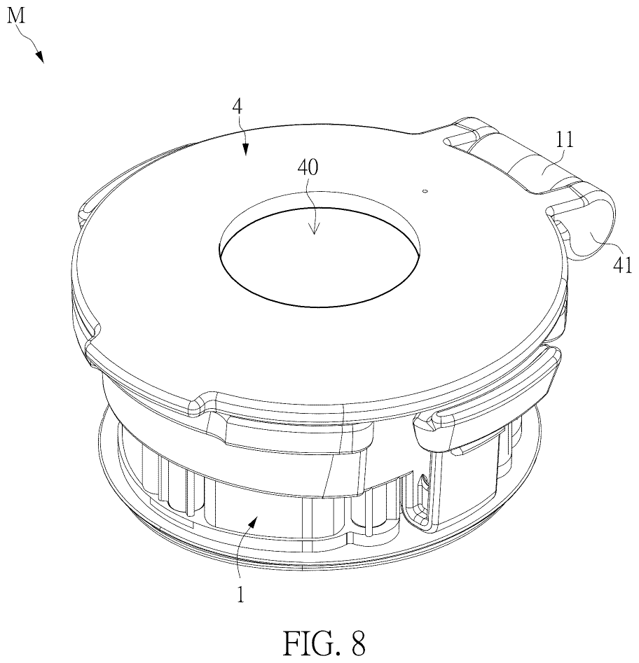

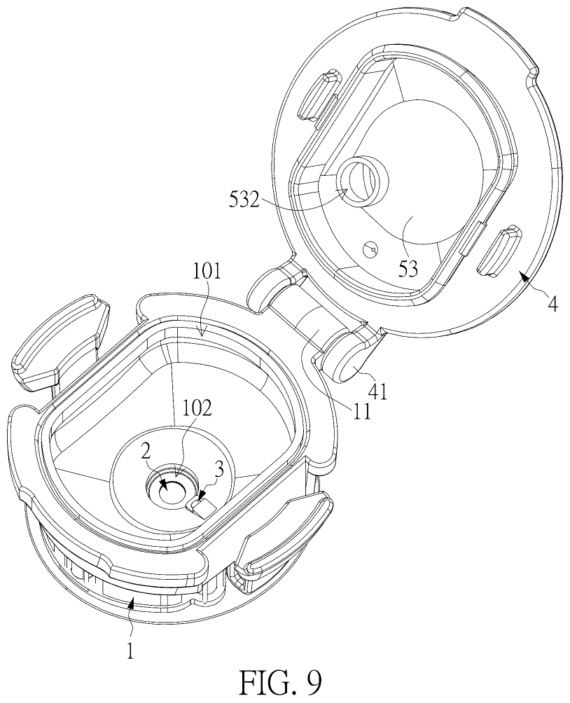

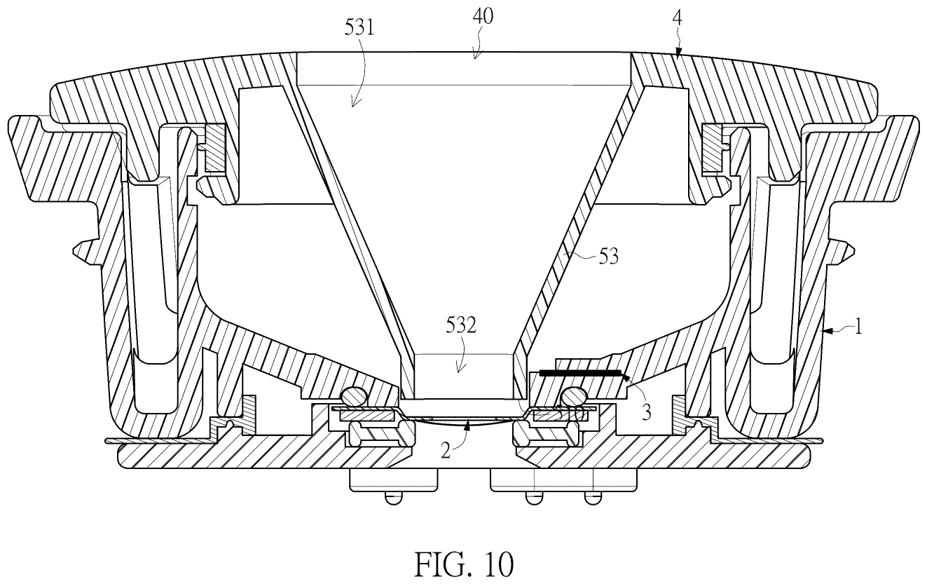

Reference is made to FIG. 8 to FIG. 10. A fourth embodiment of the present disclosure provides a liquid reservoir M, which includes a liquid storage chamber 1, a nebulizing module 2, a detection module 3, a lid 4, and a bubble blocking structure 5. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 has a penetrating hole 40. The lid 4 is pivotally connected to the liquid storage chamber 1 and covers the opening 101.

The bubble blocking structure 5 further includes a straight pipe 53. The straight pipe 53 includes a first port 531 and a second port 532. The straight pipe 53 is connected to a lower surface of the lid 4 through the first port 531. The first port 531 is in spatial communication with the penetrating hole 40. The second port 532 extends into the through hole 102. A diameter of the second port 532 is smaller than a diameter of the through hole 102. When the second port 532 extends into the through hole 102, the through hole 102 is not blocked because there is a gap between the straight pipe 53 and the through hole 102, allowing the medicinal liquid stored in the liquid storage chamber 1 to flow into the through hole 102 and be nebulized by the nebulizing module 2.

Preferably, the straight pipe 53 forms a funnel shape. The diameter of the straight pipe 53 tapers from the first port 531 to the second port 532. Therefore, the bubbles generated by the nebulizing process of the medicinal liquid are collected by the straight pipe 53. Specifically, the bubbles are adsorbed to a surface of the straight pipe 53 and move upward along the surface of the straight pipe 53, and are collected inside the straight pipe 53. In other words, the bubbles are collected and blocked by the straight pipe 53 to prevent them from covering or remaining in a detection area of the detection module 3, thereby avoiding any impact on the detection results caused by the bubbles covering or remaining in the detection area.

### Fifth Embodiment

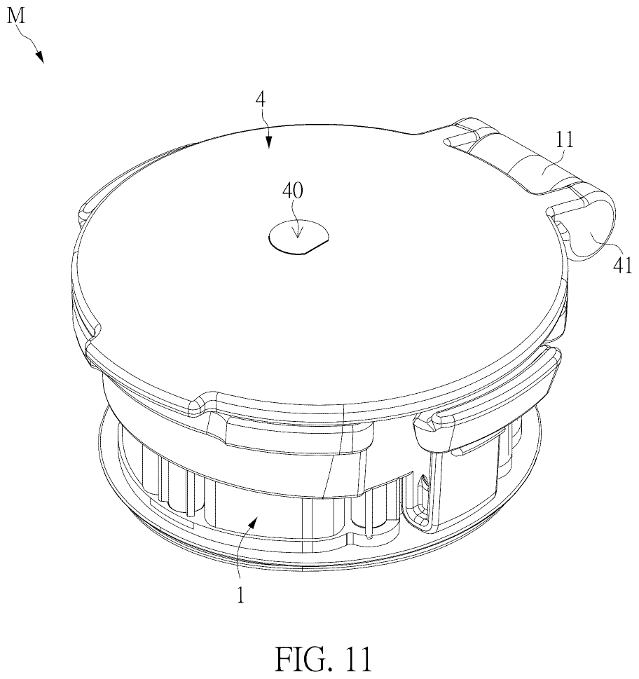

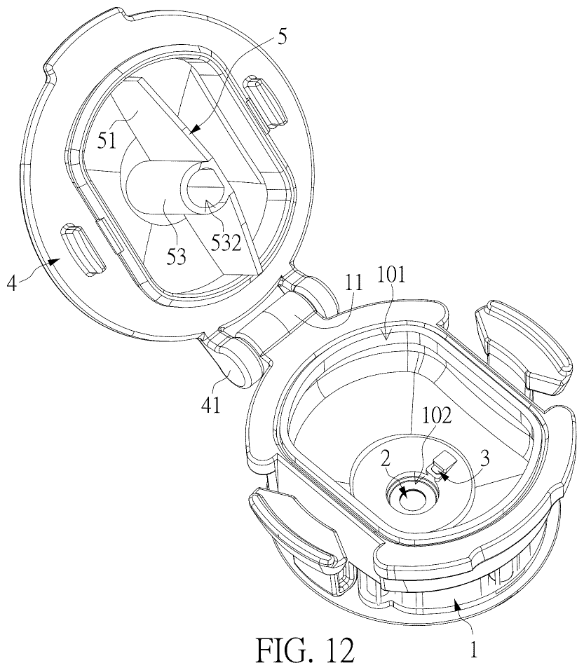

Reference is made to FIG. 11 to FIG. 13. A fifth embodiment of the present disclosure provides a liquid reservoir M, which includes a liquid storage chamber 1, a nebulizing module 2, a detection module 3, a lid 4, and a bubble blocking structure 5. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 has a penetrating hole 40 forming a circular shape. The lid 4 is pivotally connected to the liquid storage chamber 1 and covers the opening 101.

The bubble blocking structure 5 includes a partition wall 51 and a straight pipe 53. The straight pipe 53 is connected to a side of the partition wall 51 facing away from the detection module 3. The partition wall 51 is connected to a lower edge of the bubble blocking structure 5. An orthogonal projection of the partition wall 51 that is projected onto the bottom of the liquid storage chamber 1 is located at a central position of the through hole 102. The straight pipe 53 includes a first port 531 and a second port 532. The first port 531 is connected to the lid 4 and in spatial communication with the penetrating hole 40. The second port 532 extends above the through hole 102.

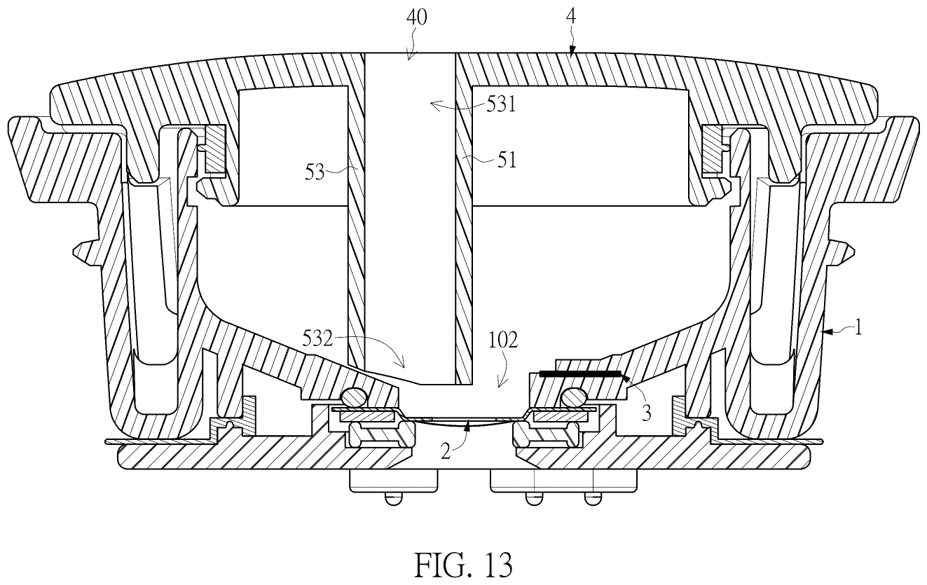

The straight pipe 53 forms a cylindrical shape. A diameter of the first port 531 is the same as a diameter of the second port 532. Since the straight pipe 53 is connected to a side of the partition wall 51, the second port 532 is not completely opposite to the through hole 102. In other words, an orthogonal projection of the straight pipe 53 that is projected onto the bottom of the liquid storage chamber 1 at least partially overlaps the through hole 102. As shown in FIG. 13, the through hole 102 and a bottom of the straight pipe 53 have a gap therebetween.

Therefore, the bubbles generated during the nebulizing process of the medicinal liquid can be collected inside the straight pipe 53 and further blocked by the partition wall 51, so as to avoid any impact on the detection results caused by the bubbles covering or remaining in a detection area of the detection module 3.

### Sixth Embodiment

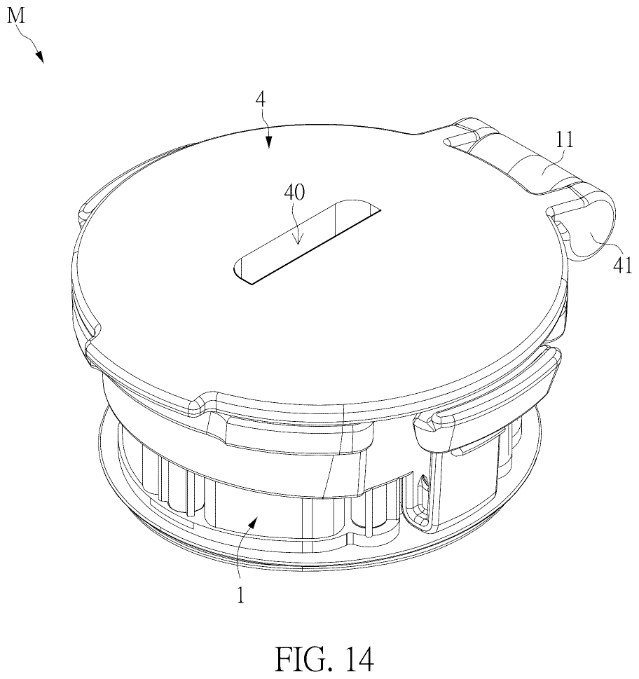

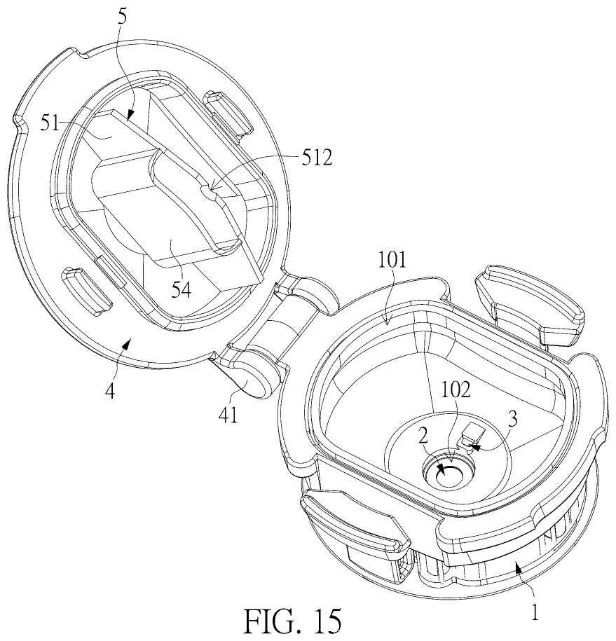

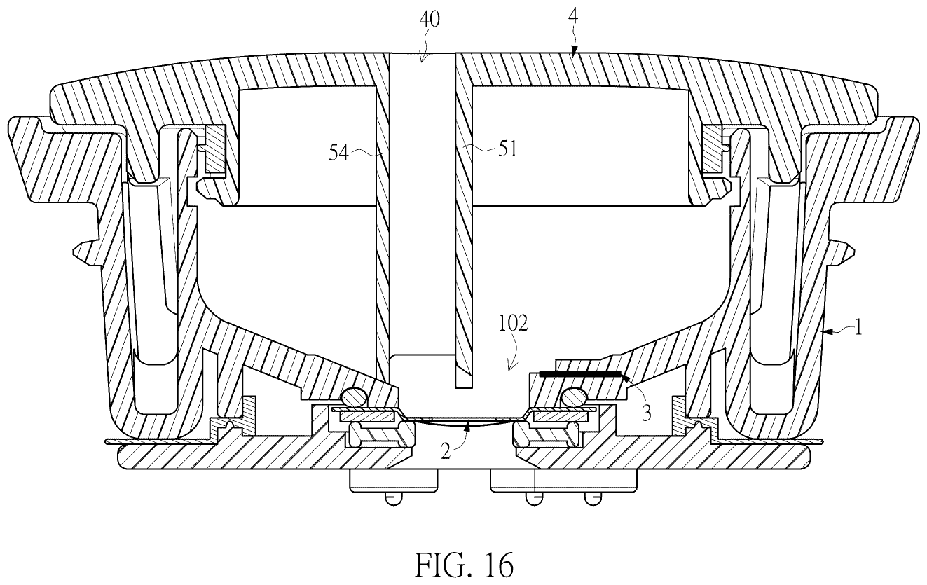

Reference is made to FIG. 14 to FIG. 16. A sixth embodiment of the present disclosure provides a liquid reservoir M, which includes a liquid storage chamber 1, a nebulizing module 2, a detection module 3, a lid 4, and a bubble blocking structure 5. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 has a penetrating hole 40 forming a flat shape. The lid 4 is pivotally connected to the liquid storage chamber 1 and covers the opening 101.

The bubble blocking structure 5 includes a partition wall 51 and an annular wall 54. The partition wall 51 is connected to a lower surface of the bubble blocking structure 5. The annular wall 54 is connected to a side of the partition wall 51 facing away from the detection module 3, and the annular wall 54 and the partition wall 51 jointly form a straight pipe that is in spatial communication with the penetrating hole 40. Because of the shape of the annular wall 54, the formed straight pipe has a flat column shape. A lower edge of the partition wall 51 is provided with a notch 512.

An orthogonal projection of the partition wall 51 that is projected onto the bottom of the liquid storage chamber 1 is located at a central position of the through hole 102. In addition, the notch 512 of the partition wall 51 and the through hole 102 have a gap therebetween, allowing the medicinal liquid stored in the liquid storage chamber 1 to flow into the through hole 102. Since the annular wall 54 is connected to a side of the partition wall 51, an orthogonal projection of the formed straight pipe that is projected onto the bottom of the liquid storage chamber 1 partially overlaps the through hole 102. Therefore, the bubbles generated during the nebulizing process of the medicinal liquid can be collected inside the straight pipe and further blocked by the partition wall 51, so as to avoid any impact on the detection results caused by the bubbles covering or remaining in a detection area of the detection module 3.

### Seventh Embodiment

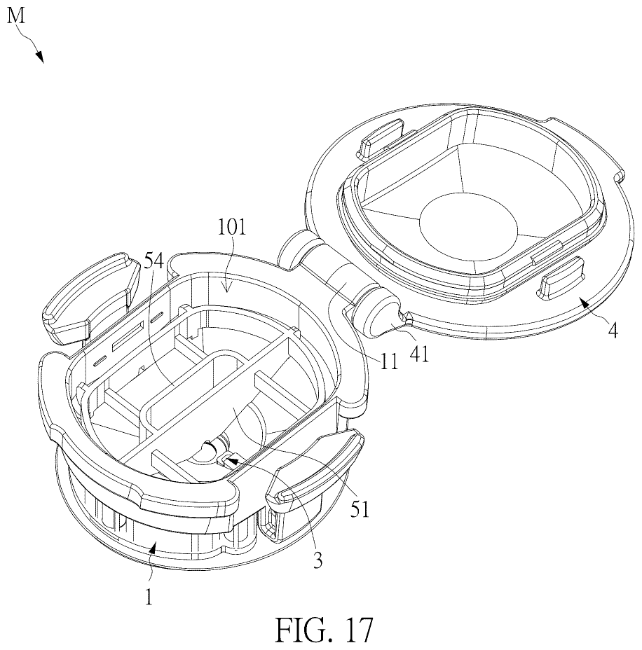

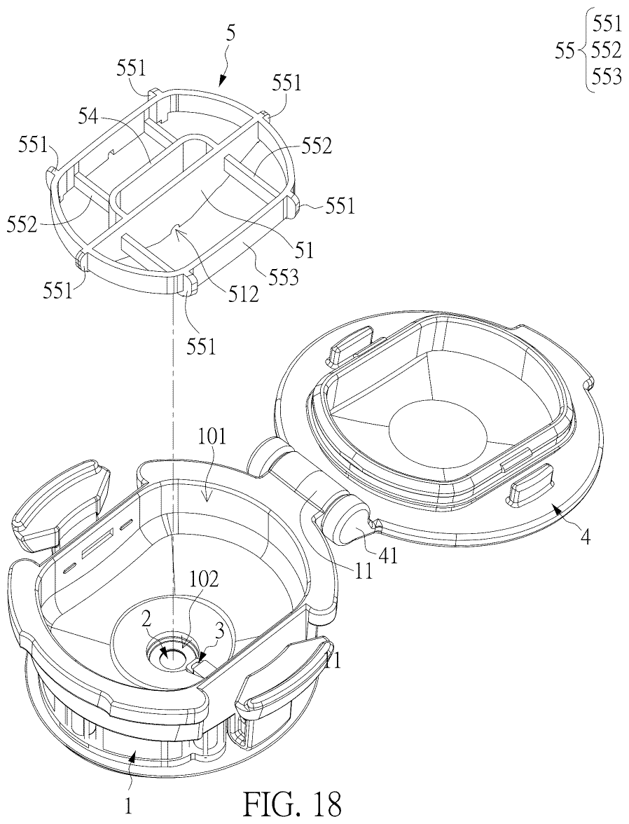

Reference is made to FIG. 17 and FIG. 18. A seventh embodiment of the present disclosure provides a liquid reservoir M, which includes a liquid storage chamber 1, a nebulizing module 2, a detection module 3, a lid 4, and a bubble blocking structure 5. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 is pivotally connected to the liquid storage chamber 1 and covers the opening 101.

The bubble blocking structure 5 includes a partition wall 51, an annular wall 54, and a holding structure 55. The annular wall 54 is connected to a side of the partition wall 51 facing away from the detection module 3. The annular wall 54 and the partition wall 51 jointly form a straight pipe. Because of the shape of the annular wall 54, the formed straight pipe forms a flat column shape. A lower edge of the partition wall 51 is provided with a notch 512. The notch 512 and the through hole 102 have a gap therebetween, allowing the medicinal liquid stored in the liquid storage chamber 1 to flow into the through hole 102. The holding structure 55 is connected to the partition wall 51 and the annular wall 54.

An orthogonal projection of the partition wall 51 that is projected onto the bottom of the liquid storage chamber 1 is located at a central position of the through hole 102. Therefore, an orthogonal projection of the straight pipe jointly formed by the partition wall 51 and the annular wall 54 that is projected onto the bottom of the liquid storage chamber 1 partially overlaps the through hole 102. Through the structural design of the partition wall 51 and the annular wall 54, the bubbles generated during the nebulizing process of the medicinal liquid can be collected inside the straight pipe and be further blocked by the partition wall 51, so as to avoid any impact on the detection results caused by the bubbles covering or remaining in a detection area of the detection module 3.

The holding structure 55 includes a plurality of protruding members 551, a plurality of connecting members 552, and a plurality of annular members 553. The annular members 553 surround outside of the partition wall 51 and the annular wall 54. Two sides of the partition wall 51 are connected to the annular members 553. Some connecting members 552 are connected between the partition wall 51 and the annular members 553, and other connecting members 552 are connected between the annular wall 54 and the annular members 553. The plurality of protruding members 551 are connected to the annular members 553 and evenly distributed around the straight pipe. Through the design of the holding structure 55, the structural strength of the bubble blocking structure 5 can be strengthened.

In the seventh embodiments, the bubble blocking structure 5 is a detachable independent component. Therefore, the bubble blocking structure 5 can be assembled into the liquid storage chamber 1, and disassembled from it to facilitate cleaning. When the bubble blocking structure 5 is assembled in the liquid storage chamber 1, the plurality of protruding members 551 abut against an inner wall of the liquid storage chamber 1 and are fixed in the liquid storage chamber 1.

### Eighth Embodiment

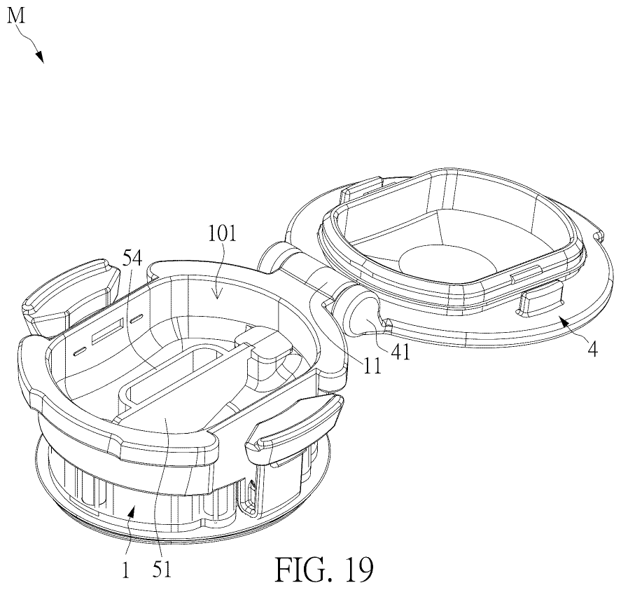

Reference is made to FIG. 19 to FIG. 21. An eighth embodiment of the present disclosure provides a liquid reservoir M, which includes a liquid storage chamber 1, a nebulizing module 2, a detection module 3, a lid 4, and a bubble blocking structure 5. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 is pivotally connected to the liquid storage chamber 1 and covers the opening 101.

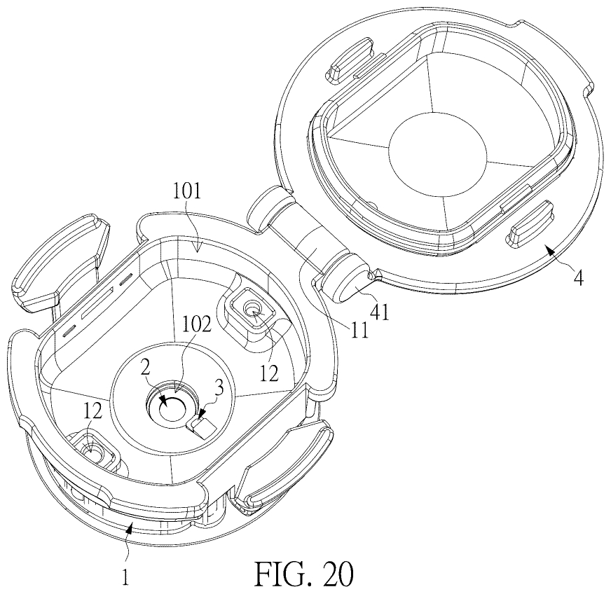

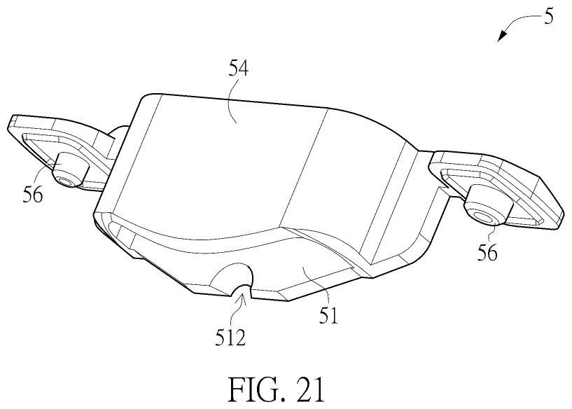

The bubble blocking structure 5 includes a partition wall 51, an annular wall 54, and two first fixing members 56. The annular wall 54 is connected to a side of the partition wall 51 facing away from the detection module 3. The annular wall 54 and the partition wall 51 jointly form a straight pipe forming a flat column shape. The two first fixing members 56 are connected to two sides of the partition wall 51, respectively. The liquid storage chamber 1 includes two second fixing members 12 therein. For example, as shown in FIGS. 20 and 21, the two first fixing members 56 can be convex posts, and the two second fixing members 12 can be concave holes. Therefore, the bubble blocking structure 5 is fixed in the liquid storage chamber 1 through the two first fixing members 56 being engaged with the two second fixing members 12 (i.e., the two convex posts are engaged with the two concave holes), respectively. A lower edge of the partition wall 51 is provided with a notch 512. The notch 512 and the through hole 102 have a gap therebetween, allowing the medicinal liquid stored in the liquid storage chamber 1 to flow into the through hole 102.

The bubble blocking structure 5 is a detachable independent component. Therefore, the bubble blocking structure 5 can be assembled into the liquid storage chamber 1, and disassembled from it to facilitate cleaning. When the bubble blocking structure 5 is assembled into the liquid storage chamber 1, the bubbles generated during the nebulizing process of the medicinal liquid can be collected inside the straight pipe 53 and further blocked by the partition wall 51, so as to avoid any impact on the detection results caused by the bubbles covering or remaining in a detection area of the detection module 3.

### Ninth Embodiment

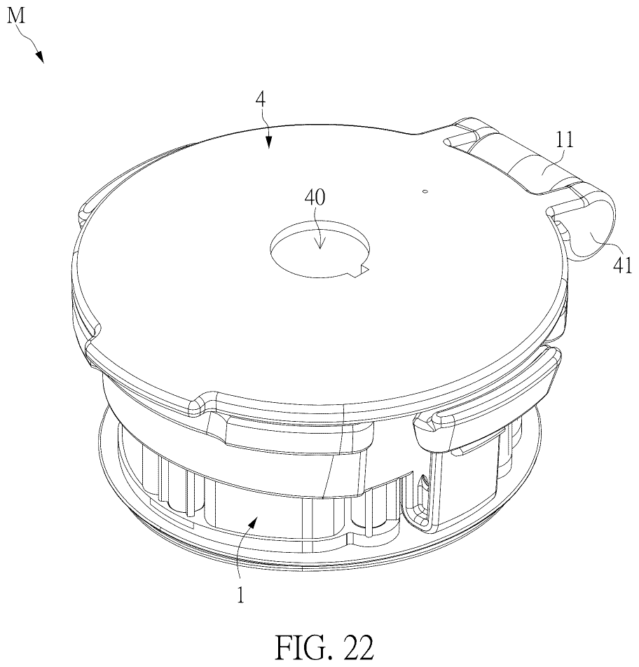

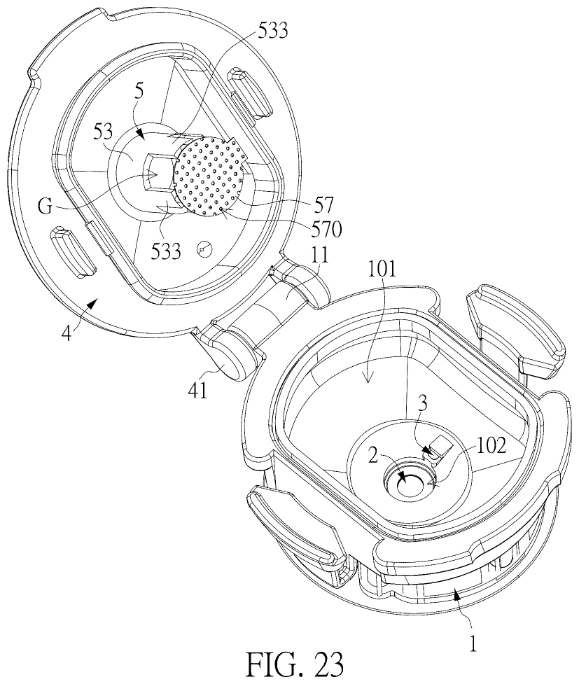

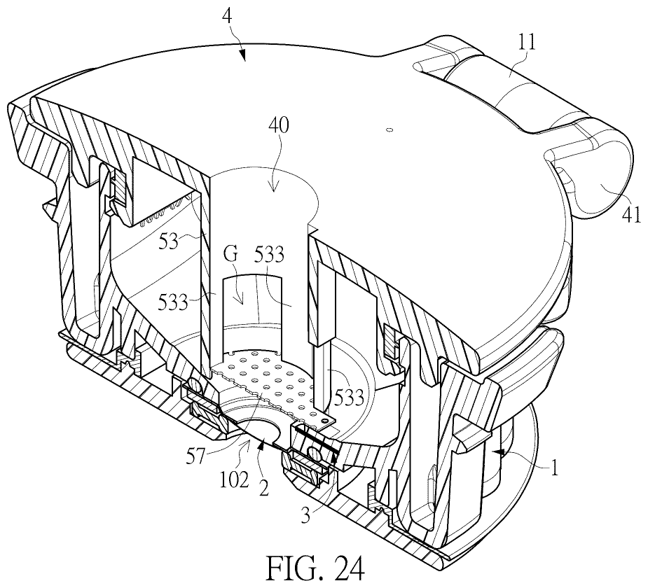

Reference is made to FIG. 22 to FIG. 24. A ninth embodiment of the present disclosure provides a liquid reservoir M, which includes a liquid storage chamber 1, a nebulizing module 2, a detection module 3, a lid 4, and a bubble blocking structure 5. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 has a penetrating hole 40. The lid 4 is pivotally connected to the liquid storage chamber 1 and covers the opening 101.

The bubble blocking structure 5 further includes a straight pipe 53 and a perforated mesh 57. The straight pipe 53 includes a plurality of connecting arms 533, and two adjacent ones of the connecting arms 533 have a gap G therebetween. The straight pipe 53 is connected to the lid 4 and in spatial communication with the penetrating hole 40. The perforated mesh 57 includes a plurality of mesh holes 570. A plurality of fixing posts (not shown in the figures) of the plurality of connecting arms 533 are inserted into some of the mesh holes 570 of the perforated mesh 57, such that the perforated mesh 57 is fixed to the straight pipe 53. Therefore, the medicinal liquid stored in the liquid storage chamber 1 can flow into the through hole 102 through the gaps G and the mesh holes 570, and be nebulized by the nebulizing module 2.

The bubbles generated during the nebulizing process of the medicinal liquid can be adsorbed to the surfaces of the mesh holes 570 and the connecting arms 533, move upward along the surfaces of the connecting arms 533, and be collected inside the straight pipe 53. Therefore, the bubbles can be blocked by the straight pipe 53 from covering or remaining in a detection area of the detection module 3, thereby avoiding any impact on the detection results caused by the bubbles covering or remaining in the detection area.

### Tenth Embodiment

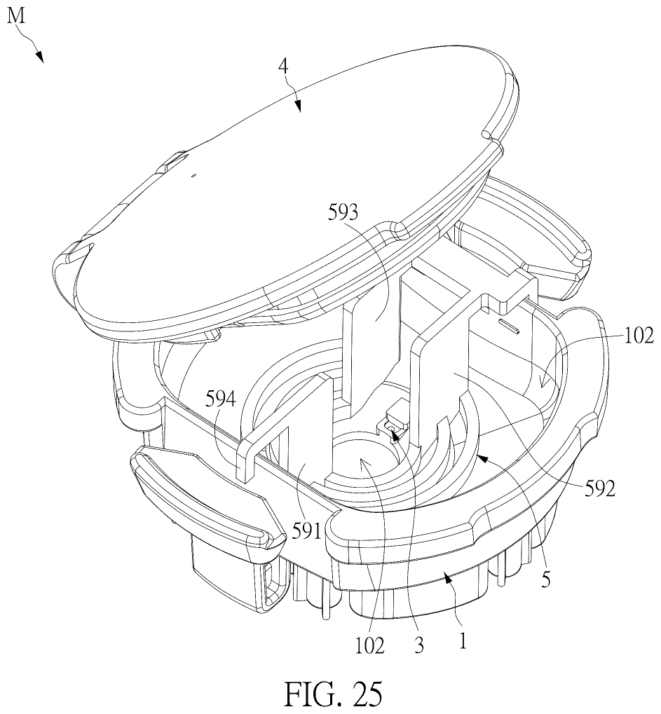

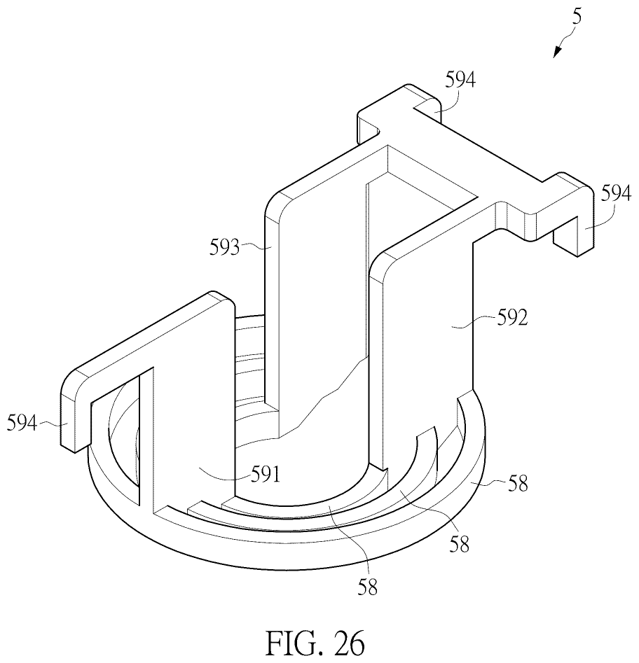

Reference is made to FIG. 25 and FIG. 26. A tenth embodiment of the present disclosure provides a liquid reservoir M, which includes a liquid storage chamber 1, a nebulizing module 2, a detection module 3, a lid 4, and a bubble blocking structure 5. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 is pivotally connected to the liquid storage chamber 1 and covers the opening 101.

The bubble blocking structure 5 includes a plurality of annular members 58, a first holding member 591, a second holding member 592, and a third holding member 593. The plurality of annular members 58 jointly form a structure of concentric circles. The first holding member 591 is connected to and extends between the plurality of annular members 58. One end of each of the annular members 58 is connected to the second holding member 592, another end of the annular members 58 is connected to the third holding member 593. There is an accommodating space between the second holding member 592 and the third holding member 593.

The bubble blocking structure 5 is a detachable independent component which can be assembled into the liquid storage chamber 1, and disassembled from it to facilitate cleaning. One end of each of the first holding member 591, the second holding member 592, and the third holding member 593 away from the annular members 58 forms a hook 594. As shown in FIG. 25, the bubble blocking structure 5 is disposed in the liquid storage chamber 1 by the hook 594 being fastened to an edge of the opening 101, and the plurality of annular members 58 surrounds the through hole 102. In other words, the through hole 102 is located at the center of the concentric circle structure. When the bubble blocking structure 5 is disposed in the liquid storage chamber 1, the detection module 3 is located between the second holding member 592 and the third holding member 593.

Through the structural design of the bubble blocking structure 5 and the annular members 58, the bubbles that are generated during the nebulizing process of the medicinal liquid can adhere sequentially from the outside to the inside between the plurality of annular members 58 as the liquid level decreases, so as to avoid any impact on the detection results caused by the bubbles covering or remaining in a detection area of the detection module 3.

### Eleventh Embodiment

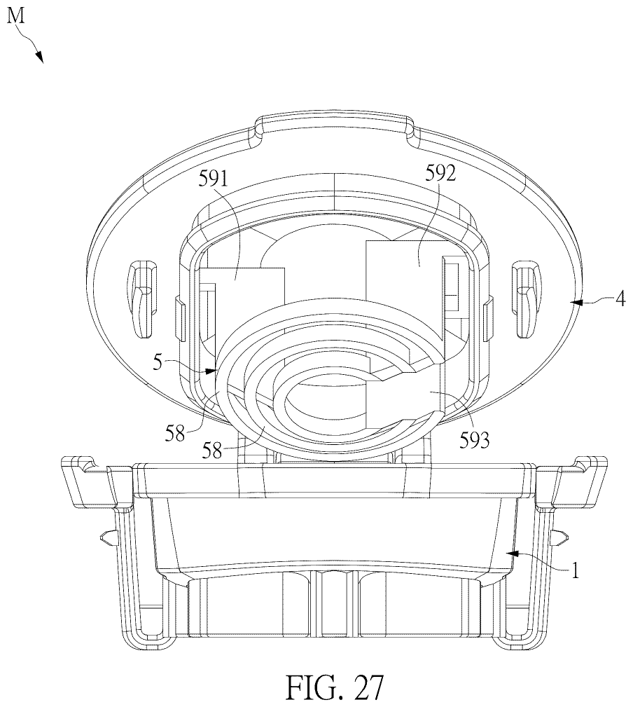

Reference is made to FIG. 27. An eleventh embodiment of the present disclosure provides a liquid reservoir M, which includes a liquid storage chamber 1, a nebulizing module 2, a detection module 3, a lid 4, and a bubble blocking structure 5. The structures of the liquid storage chamber 1, the nebulizing module 2, the detection module 3, and the lid 4 in the liquid reservoir M of the eleventh embodiments are similar to those of the tenth embodiment. Therefore, the eleventh embodiment can be illustrated in conjunction with FIG. 25. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 is pivotally connected to the liquid storage chamber 1 and covers the opening 101.

The bubble blocking structure 5 includes a plurality of annular members 58, a first holding member 591, a second holding member 592, and a third holding member 593. The plurality of annular members 58 jointly form a structure of concentric circles. The first holding member 591 is connected to and extends between the plurality of annular members 58. One end of each of the annular members 58 is connected to the second holding member 592, another end of the annular members 58 is connected to the third holding member 593. There is an accommodating space between the second holding member 592 and the third holding member 593.

As shown in FIG. 27, in the eleventh embodiment, the first holding member 591, the second holding member 592, and the third holding member 593 of the bubble blocking structure 5 are connected to a lower surface of the lid 4; therefore, the bubble blocking structure 5 can be placed inside the liquid storage chamber 1 as the lid 4 covers the liquid storage chamber 1. When the bubble blocking structure 5 is disposed in the liquid storage chamber 1, the plurality of annular members 58 surrounds the through hole 102, the through hole 102 is located at the center of the concentric circle structure, and the detection module 3 is located between the second holding member 592 and the third holding member 593.

### Twelfth Embodiment

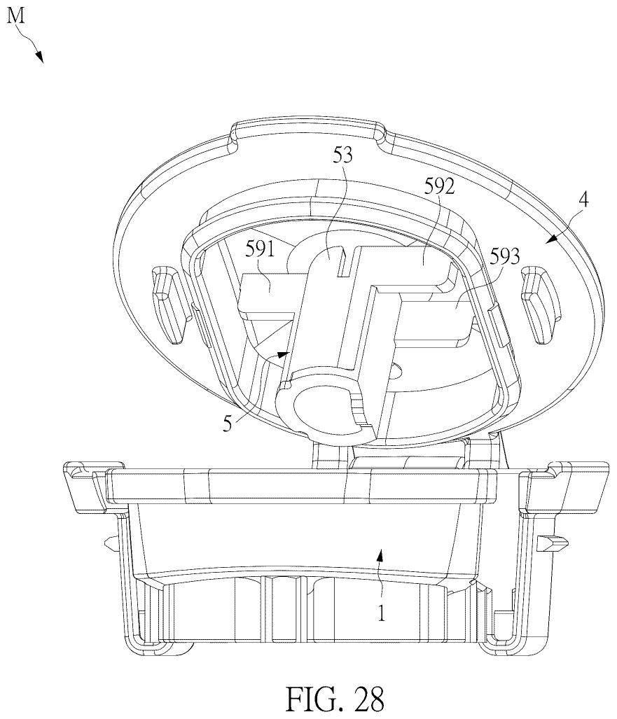

Reference is made to FIG. 28. A twelfth embodiment of the present disclosure provides a liquid reservoir M, which includes a liquid storage chamber 1, a nebulizing module 2, a detection module 3, a lid 4, and a bubble blocking structure 5. The structures of the liquid storage chamber 1, the nebulizing module 2, the detection module 3, and the lid 4 in the liquid reservoir M of the twelfth embodiments are similar to those of the first embodiment. Therefore, the twelfth embodiment can be illustrated in conjunction with FIG. 2. A top of the liquid storage chamber 1 has an opening 101, and a bottom of the liquid storage chamber 1 has a through hole 102. The nebulizing module 2 is disposed in the through hole 102. The detection module 3 is disposed within the liquid storage chamber 1, and the detection module 3 is adjacent to the through hole 102. The lid 4 is pivotally connected to the liquid storage chamber 1 and covers the opening 101.

The bubble blocking structure 5 includes a straight pipe 53, a first holding member 591, a second holding member 592, and a third holding member 593. The straight pipe 53 forms a cylindrical shape. The straight pipe 53 is located directly above and in spatial communication with the through hole 102. The first holding member 591, the second holding member 592, and the third holding member 593 are connected to the straight pipe 53. In addition, the first holding member 591, the second holding member 592, and the third holding member 593 are connected to a bottom of the lid 4; therefore, the bubble blocking structure 5 can be placed inside the liquid storage chamber 1 as the lid 4 covers the liquid storage chamber 1.

### Thirteenth Embodiment

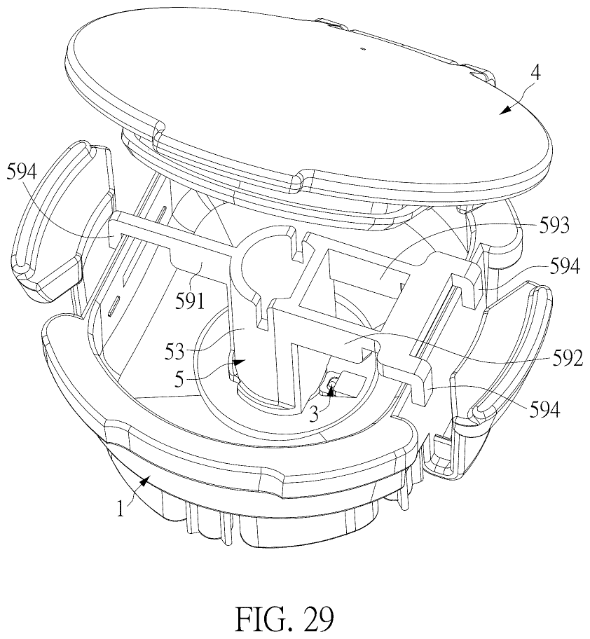

Reference is made to FIG. 29. A thirteenth embodiment of the present disclosure provides a liquid reservoir M, which includes a liquid storage chamber 1, a nebulizing module 2, a detection module 3, a lid 4, and a bubble blocking structure 5. The structures of the liquid storage chamber 1, the nebulizing module 2, the detection module 3, and the lid 4 in the liquid reservoir M of the thirteenth embodiments are similar to those of the first embodiment. Therefore, the twelfth embodiment can be illustrated in conjunction with FIG. 2. The bubble blocking structure 5 includes a straight pipe 53, a first holding member 591, a second holding member 592, and a third holding member 593. The straight pipe 53 is located directly above and in spatial communication with the through hole 102. The first holding member 591, the second holding member 592, and the third holding member 593 are connected to the straight pipe 53.

Furthermore, one end of each of the first holding member 591, the second holding member 592, and the third holding member 593 away from the annular members 58 forms a hook 594. As shown in FIG. 29, the bubble blocking structure 5 is disposed in the liquid storage chamber 1 by the hook 594 being fastened to an edge of the opening 101.

Comparing the liquid reservoir M of the thirteenth embodiment with that of the twelfth embodiment, the main differences reside in the first holding member 591, the second holding member 592, and the third holding member 593. In the twelfth embodiment, the first holding member 591, the second holding member 592, and the third holding member 593 are connected to a lower edge of the lid 4. In the thirteenth embodiment, the first holding member 591, the second holding member 592, and the third holding member 593 are fastened to an edge of the opening 101 through the hooks 594.

Therefore, the bubbles that are generated during the nebulizing process of the medicinal liquid are collected by the straight pipe 53. Specifically, the bubbles are adsorbed to a surface of the straight pipe 53 and move upward along the surface of the straight pipe 53, and can be collected inside the straight pipe 53. In other words, the bubbles are blocked by the straight pipe 53 to prevent them from covering or remaining in a detection area of the detection module 3, thereby avoiding any impact on the detection results caused by the bubbles covering or remaining in the detection area.

### Beneficial Effects of the Embodiments

In the liquid reservoir M provided by the present disclosure, by the bubble blocking structure 5 being disposed within the liquid storage chamber 1, the bubble blocking structure 5 can be used to either adhere to or attract bubbles through surface properties, or to block the bubbles through structural design. When the bubbles are generated during the nebulizing process of the medicinal liquid, the bubble blocking structure 5 collects and isolates the bubbles to prevent them from covering or remaining in the detection area of the detection module 3, thereby avoiding any impact on the detection results caused by the bubbles covering or remaining in the detection area.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A liquid reservoir, comprising:
a liquid storage chamber having an opening, and a bottom of the liquid storage chamber having a through hole;
a nebulizing module disposed in the through hole;
a detection module disposed within the liquid storage chamber and being adjacent to the through hole;
a lid disposed on the liquid storage chamber and covering the opening; and
a bubble blocking structure disposed within the liquid storage chamber, wherein an orthogonal projection of the bubble blocking structure that is projected onto the bottom of the liquid storage chamber at least partially overlaps the through hole.

2. The liquid reservoir according to claim 1, wherein the bubble blocking structure includes a partition wall connected to the lid, and the orthogonal projection of the bubble blocking structure that is projected onto the bottom of the liquid storage chamber is located at a central position of the through hole.

3. The liquid reservoir according to claim 2, wherein a lower edge of the bubble blocking structure is lower than the detection module and higher than the through hole.

4. The liquid reservoir according to claim 1, wherein the bubble blocking structure includes a partition wall connected to the lid, and the orthogonal projection of the bubble blocking structure that is projected onto the bottom of the liquid storage chamber is located between the detection module and a central position of the through hole.

5. The liquid reservoir according to claim 4, wherein a lower edge of the partition wall includes a convex portion, and the convex portion extends into the through hole and abuts against an inner edge of the through hole.

6. The liquid reservoir according to claim 4, wherein the bubble blocking structure further includes a first fixing rib and two second fixing ribs that are connected to opposite sides of the partition wall; and wherein the first fixing rib is disposed on the side of the partition wall facing the detection module.

7. The liquid reservoir according to claim 6, wherein the two second fixing ribs are orthogonally projected onto the bottom of the liquid storage chamber to form two second orthogonal projections, and the through hole is located between the two second orthogonal projections.

8. The liquid reservoir according to claim 2, wherein the bubble blocking structure further includes a straight pipe that is connected to a side of the partition wall facing away from the detection module; and wherein the lid has a penetrating hole, the straight pipe includes a first port and a second port, the first port is connected to the lid and in spatial communication with the penetrating hole, and the second port extends above the through hole.

9. The liquid reservoir according to claim 8, wherein the straight pipe forms a cylindrical shape.

10. The liquid reservoir according to claim 1, wherein the bubble blocking structure includes a straight pipe, the straight pipe includes a first port and a second port, the lid has a penetrating hole, the first port is connected to the lid and in communication with the penetrating hole, the second port extends into the through hole, and a diameter of the straight pipe tapers from the first port to the second port.

11. The liquid reservoir according to claim 1, wherein the bubble blocking structure includes a partition wall and an annular wall, a lower edge of the partition wall is provided with a notch, the annular wall is connected to a side of the partition wall facing away from the detection module, and the annular wall and the partition wall jointly form a straight pipe.

12. The liquid reservoir according to claim 11, wherein the straight pipe has a flat column shape, and a lower edge of the partition wall is provided with a notch.

13. The liquid reservoir according to claim 11, wherein the bubble blocking structure further includes a holding structure that is connected to the annular wall and the partition wall, the holding structure includes a plurality of protruding members, and the plurality of protruding members are evenly distributed around the straight pipe and abut against an inner wall of the liquid storage chamber.

14. The liquid reservoir according to claim 11, wherein the bubble blocking structure further includes two first fixing members that are connected to two sides of the partition wall, respectively, and the liquid storage chamber includes two second fixing members therein; and wherein the bubble blocking structure is fixed in the liquid storage chamber through the two first fixing members being engaged with the two second fixing members, respectively.

15. The liquid reservoir according to claim 1, wherein the bubble blocking structure further includes a straight pipe, the lid has a penetrating hole, the straight pipe includes a plurality of connecting arms, two adjacent ones of the connecting arms have a gap therebetween, and the straight pipe is connected to the lid and in communication with the penetrating hole.

16. The liquid reservoir according to claim 15, wherein the bubble blocking structure further includes a perforated mesh that is disposed above the through hole, the perforated mesh includes a plurality of mesh holes, the plurality of connecting arms have a plurality of fixing members, respectively, and the plurality of connecting arms are inserted into some of the mesh holes through the plurality of fixing members.

17. A liquid reservoir, comprising:
a liquid storage chamber having an opening, and a bottom of the liquid storage chamber having a through hole;
a nebulizing module disposed in the through hole;
a detection module disposed within the liquid storage chamber and being adjacent to the through hole;
a lid disposed on the liquid storage chamber and covering the opening; and
a bubble blocking structure disposed within the liquid storage chamber, wherein the bubble blocking structure includes a plurality of annular members surrounding the through hole and jointly forming a structure of concentric circles.

18. The liquid reservoir according to claim 17, wherein the bubble blocking structure further includes a first holding member that is connected to and extends between the plurality of annular members.

19. The liquid reservoir according to claim 18, wherein the bubble blocking structure further includes a second holding member and a third holding member, one end of each of the annular members is connected to the second holding member, another end of the annular members is connected to the third holding member, and the detection module is located between the second holding member and the third holding member.

20. The liquid reservoir according to claim 19, wherein one end of each of the first holding member, the second holding member, and the third holding member away from the annular members forms a hook, and the hook is fastened to an edge of the opening.

21. The liquid reservoir according to claim 19, wherein the first holding member, the second holding member, and the third holding member are connected to a bottom of the lid.

22. A liquid reservoir, comprising:
a liquid storage chamber having an opening, and a bottom of the liquid storage chamber having a through hole;
a nebulizing module disposed in the through hole;
a detection module disposed within the liquid storage chamber and being adjacent to the through hole;
a lid disposed on the liquid storage chamber and covering the opening; and
a bubble blocking structure disposed within the liquid storage chamber, wherein the bubble blocking structure includes a straight pipe, and the straight pipe is located directly above the through hole.

23. The liquid reservoir according to claim 22, wherein the bubble blocking structure further includes a first holding member, a second holding member, and a third holding member that are connected to the straight pipe.

24. The liquid reservoir according to claim 23, wherein one end of each of the first holding member, the second holding member, and the third holding member away from the annular members forms a hook, and the hook is fastened to an edge of the opening.

25. The liquid reservoir according to claim 23, wherein the first holding member, the second holding member, and the third holding member are connected to a bottom of the lid.
